(19) United States
(12) Patent Application Publication
(10) Pub. No.: US 2016/0030684 A1
(43) Pub. Date: Feb. 4, 2016

(54) LUER ADAPTER FITTING AND DEVICES THEREWITH

(71) Applicant: SMITHS MEDICAL ASD, INC., Rockland, MA (US)

(72) Inventor: Charles Pratt, Stoddard, NH (US)

(73) Assignee: SMITHS MEDICAL ASD, INC., Rockland, MA (US)

(21) Appl. No.: 14/808,118

(22) Filed: Jul. 24, 2015

Related U.S. Application Data
(60) Provisional application No. 62/030,648, filed on Jul. 30, 2014.

(51) Int. Cl. A61M 5/34; A61J 1/14; A61M 39/10

(52) U.S. Cl. CPC A61M 5/345; A61M 39/10; A61J 1/1481; A61M 5/347; A61M 2039/1033; A61M 2039/1066; A61M 2039/1077; A61M 2039/1094; A61M 2039/1027; A61M 2207/00

(57) ABSTRACT

A luer adapter fitting has a luer at one end and an elongate cylindrical insert at another end. The insert has a plurality of longitudinal ribs spatially formed about the insert. Each of the ribs has a sharp longitudinal edge. A needle extends from the end of the insert away from the luer. The luer adapter fitting is press-fitted to an internally threaded through bore at the neck of a tube holder. The circumferential dimension formed by the plurality of ribs is larger than the opening of the threaded through bore, so that when the insert is press-fitted to the through bore, the longitudinal sharp edges of the ribs would score the threads at the through bore to prevent relative rotation between the fitting and the holder. A flange at the insert snap catches onto the interior shoulder of the holder to fixedly couple the insert to the holder.

### FIELD OF THE INVENTION

The present invention is directed to blood collection or fluid transfer devices with tube holders.

There are a number of types of blood collection devices being marketed by the assignee of the instant invention. One is being sold by the assignee of the instant invention under the trade name Venipuncture Needle-Pro®. This device has a vacuum tube holder, also referred to as a vacutainer holder, that has an internally threaded neck at one receptacle end and an opening at the other reception end. A double ended needle is coupled to the holder by means of a threaded needle hub threadingly mating with the internally threaded neck of the tube holder. To draw blood from the patient, after the patient end of the needle has been inserted to the appropriate vein of the patient, a vacuum tube is inserted into the open reception end of the holder with the septum that seals the top of the vacuum tube being pierced by the tube end of the double ended needle so that a fluid path is established between the vein of the patient and the interior of the vacuum tube. Due the vacuum in the vacuum tube, blood is drawn into the vacuum tube from the patient.

A second blood collection device being sold by the assignee of the instant invention is marketed under the mark Saf-T Holder®. The Saf-T Holder® is a tube holder that also has an opening at a reception end to receive a vacuum tube. But instead of an internally threaded neck, the patient end of the Saf-T Holder® has a luer connector or simply luer, either male or female, that connects with a counterpart luer of a winged needle or the needle of a Saf-T Wing® Blood Collection & Infusion Set sold by the assignee of the instant invention. The receptacle end of the Saf-T Holder® is therefore different from the receptacle end, or neck, of the vacutainer holder.

The cost of manufacturing would be substantially reduced if the same tube holder could be used for the male and female Saf-T Holders®, vacuum tube holders and the vacutainer holders of Venipuncture Needle-Pro®.

### BACKGROUND OF THE INVENTION

To eliminate the need for specially designed vacuum tube holders for the Saf-T Wing® devices, an adapter fitting is designed to have a luer connector, or luer, at one end and an insert extending from the luer to another end wherefrom a needle extends. The needle is covered by an elastomeric or rubber shroud so that the tip of the needle is not exposed. The insert is designed to have a plurality of ribs that are spatially provided circumferentially about the elongate cylindrical insert. The respective edges of the ribs form a circumference or a circumferential dimension that has a given diameter sized or dimensioned to be slightly larger than the diameter of the opening formed by the internally threaded neck portion of a vacuum tube holder, for example the vacutainer holder as discussed above. The ribs have relatively longitudinal sharp edges so that when the body of the fitting is coupled to the neck of the vacuum tube holder, with the insert press-fitted into the neck of the holder, the ribs would score or cut into the internal threads along the length of the neck due to the plasticity or elasticity of the internal threads of the holder. Once fully inserted, the fitting and the holder are not rotatable relative to each other due to the scoring of the threads by the ribs. Further, given that the ribs are forcefully press fitted to score the internal threads at the neck of the holder, the fitting is frictionally held to the neck of the holder.

There is further provided at the end of the insert away from the luer a fully or partially circumferential flange or extension(s) that, when the fitting is fully press-fitted into the neck of the holder, acts as a catch to latch onto the internal shoulder of the holder where the neck joins the main body of the holder. This is possible since both the fitting and the holder are made of medical plastics material, for example polyurethane so that the inherent elasticity of the plastics material enables the flange to be compressed while being press-fitted into the neck and then snaps back to its original shape to latch onto the internal shoulder of the holder once the flange exits the neck. as a result, the fitting is fixedly or non-removably coupled to the holder.

### SUMMARY OF THE INVENTION

The instant invention is directed to a fitting adapted to be attached to a fluid store holder having a cylindrical body including one end with a neck having a threaded through bore and an other end having an opening adapted to receive a fluid store. The fitting comprises a body having a luer at one end and an elongate cylindrical insert extending away from the luer to an other end. A plurality of ribs are spatially formed circumferentially about the insert with each rib extending at least a given length along the insert. The circumferential dimension formed by the plurality of ribs is larger than the internal circumference of the threaded through bore of the holder so that when the insert is press fitted to the through bore, the ribs would score the threads along the through bore to prevent relative rotation between the fitting and the holder.

A catch may be provided at the other end of the insert, possibly in the form of a flange or extensions to engage with the internal shoulder that forms at the base of the through bore along the neck of the fluid store holder so that once the fitting is fully inserted to the through bore, the catch would latch onto the internal shoulder at the base of the neck, or a space provided thereat, to prevent the fitting from being removed from the fluid store holder, i.e., to fixedly couple the fitting to the holder.

The instant invention is also directed to an apparatus that has a fluid store holder having a cylindrical body including a receptacle one end with a neck having a threaded through bore and a receptive other end having an opening adapted to receive a fluid store and a fitting coupled to the neck of the tube holder. The fitting includes a luer at one end and an elongate cylindrical insert extending away from the luer to an other end, a plurality of ribs spatially formed circumferentially about the insert with each rib extending longitudinally along at least a given length of the insert, the circumferential dimension formed by the plurality of ribs being larger than the internal circumference formed by the threaded through bore, the insert press fitted to the through bore with the ribs scoring or cutting into the threads along the through bore to prevent relative rotation between the connector and the holder. The interaction between the ribs and the threaded through bore may also frictionally maintain the fitting to the neck of the holder.

The instant invention is further directed to a method comprising the steps of:

providing a fluid store holder having a cylindrical body including one end with a neck having a threaded through bore and an other end having an opening adapted to receive a fluid store;

providing a fitting including a luer at one end and an elongate cylindrical insert extending away from the one end to an other end, a plurality of ribs spatially formed circumferentially about the insert with each rib extending longitudinally along at least a given length of the insert, the circumferential dimension formed by the plurality of ribs being larger than the inner circumference of the threaded through bore at the neck of the holder; and

inserting the fitting to the through bore so that the ribs score the threads along the through bore to prevent relative rotation between the fitting and the holder so that the luer at the one end of the fitting is adapted to receive a counterpart luer.

It is therefore an objective of the instant invention to provide a luer adapter fitting that can attach a male or a female luer to a vacuum tube holder.

It is another objective of the instant invention to eliminate the need to manufacture special male and female holders for winged needle devices.

It is yet another objective of the instant invention to reduce the cost of manufacturing blood drawing or fluid transfer devices by using the same vacuum tube holder for different types of blood drawing or fluid transfer devices.

### BRIEF DESCRIPTION OF THE FIGURES

The above-mentioned objectives and advantages of the present invention will be apparent and the invention itself will be best understood by reference to the following description of the invention taken in conjunction with the accompanying drawings, wherein:

FIG. 1 illustrates a Venipuncture Needle-Pro® product of the assignee's and particularly illustrates the vacutainer holder that may be used as the vacuum tube holder of the instant invention;

FIG. 2 illustrates the assignee's Saf-T Holders® having male and female luers;

FIG. 3 shows a male luer adapter fitting of the instant invention;

FIG. 4 shows a female luer adapter fitting of the instant invention;

FIG. 5 illustrates an embodiment of the instant invention where a male luer adapter fitting as shown in FIG. 3 is in alignment along a longitudinal axis with a vacuum tube holder; and

FIG. 6 is an enlarged cross-sectional view that shows a male luer adapter fitting coupled to the neck of the vacuum tube holder to form an inventive blood drawing device that has a male luer adapter fitting.

### DETAILED DESCRIPTION OF THE INVENTION

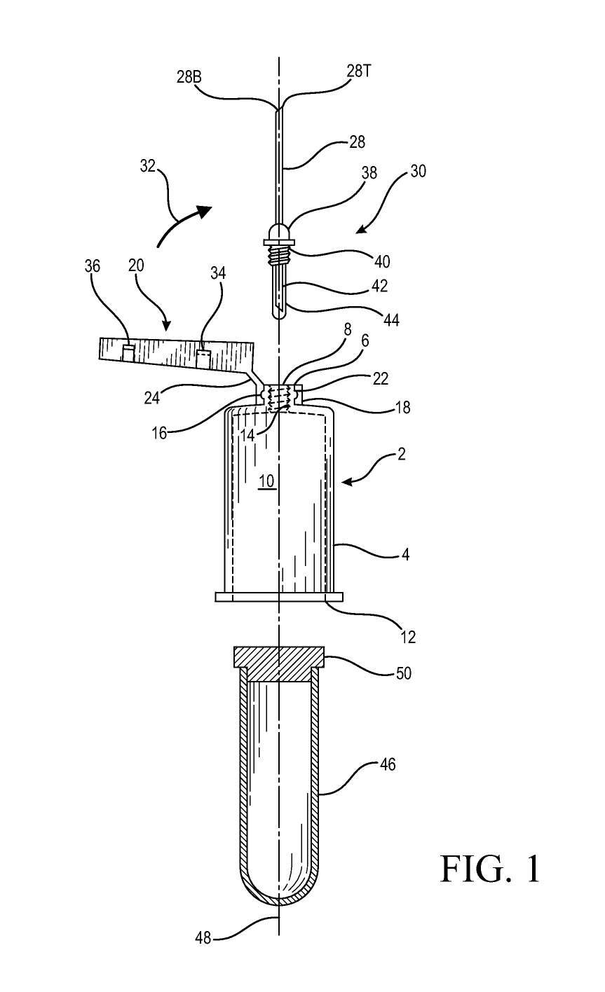

FIG. 1 shows the Venipuncture Needle-Pro® product that is being sold by the assignee of the instant invention. FIG. 1 is described in U.S. Pat. No. 5,277,311, the disclosure of which being incorporated by reference to the disclosure of the instant application.

In particular, FIG. 1 shows a vacuum tube holder 2, that is also known in the industry as a vacutainer holder. Holder 2 has a hollow cylindrical body 4 and a receptacle end, or neck 6, integrally extending from body 4 at one end. An aperture extends from opening 8 of neck 6 to cavity 10 of body 4. An opening 12 at the receptive other end of body 4 provides passage for a fluid store, for example the shown vacuum tube 46, into cavity 10 of holder 2. The inner circumferential wall of neck 6 is threaded by threads 14 to form a threaded through bore or aperture.

Formed around the outer circumference of neck 6 is a circumferential protuberance, or boss 16. A base or collar 18 of a safety sheath or housing 20 is fitted, via an internal circumferential groove 22 at base 18, about neck 6. Base 18 may be hard pressed onto neck 6 for rotatably mating its internal groove 22 to external boss 16 of neck 6. The respective dimensions of boss 16 and internal groove 22, and neck 6 and base 18, are such that base 18 is held frictionally against neck 6 such that the former is rotatable about the latter only by force, so that once base 18 has been rotated to a certain orientation about neck 6, it will stay at that orientation until additional torque force is exerted thereagainst.

Connected to base 18, via a flexible hinge 24, is a needle protection housing 20 having an elongated slot 26 (not shown) through which a needle or cannula, such as 28 of the double-ended needle assembly 30, passes when housing 20 is pivoted toward the longitudinal axis 48 of cannula 28 as per shown by directional arrow 32. Integrally formed within housing 20 is a lock mechanism that may include hooks 34 and 36. The hooks would grasp cannula 28 to prevent cannula 28 from being re-exposed, once housing 20 has been pivoted to envelop the same.

As further shown, double-ended needle assembly 30 is coupled to neck 6 by threadingly mating the external threads 40 at its needle hub 38 with the internal threads 14 at the threaded aperture or through bore of neck 6. In addition to cannula 28, which is used to prick or insert to a vein of the patient, needle assembly 30 has an opposed cannula 42 enveloped by an elastomeric or rubber shroud 44. Once needle assembly 30 is threadingly mated with neck 6, the shrouded cannula 42 is extended into cavity 10 of holder 2. When vacuum tube 46 is inserted through opening 12 into cavity 10 of body 2, its septum 50 would push back the shroud 44 and be pierced by cannula 42. A through passage is thus established between cannula 28, at its tip 28T which presumably has been inserted into the vein of the patient, to the inside cavity of tube 46, where the vacuum inside tube 46 causes blood to be withdrawn from the patient into tube 46. The vacuum tube holder 2 shown in FIG. 1, prior to the instant invention, is used exclusively with a double end needle assembly, such as 30, that has a threaded hub.

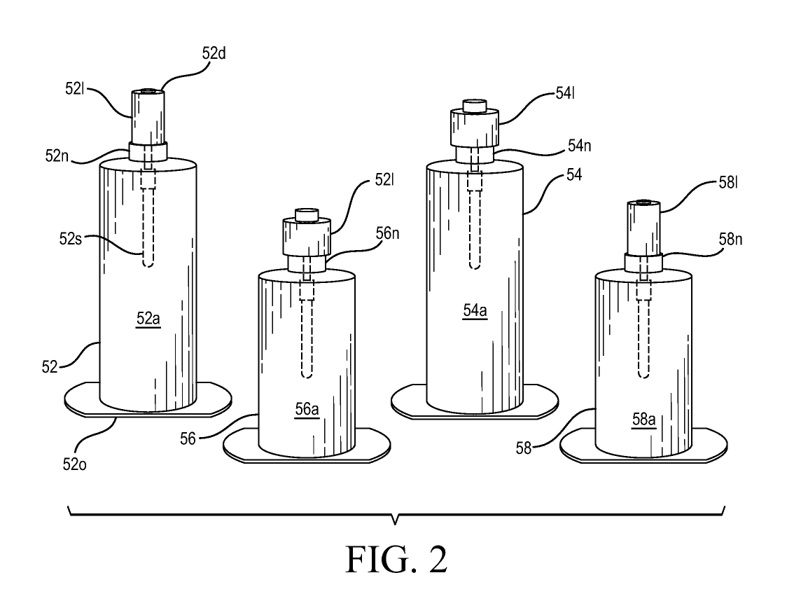

FIG. 2 shows Saf-T Holders® that are being sold by the assignee of the instant invention. In particular, the Saf-T Holders® 52, 54, 56 and 58 are specially manufactured fluid store holders that have receptacle ends in the form of luer connectors, or simply luers that may be manufactured in accordance with ISO (International Organization for Standardization) Standards ISO 594-1 and ISO 594-2. In the case of holders 52 and 54 which have longer cylindrical bodies 52a and 54a, respectively, a female luer 52l is shown to extend from neck 52n that integrally extends from holder body 52a and a male luer 54l is shown to extend from neck 52n that integrally extends from holder body 54a. Female luer 52l has a distal end 52d for accepting a counterpart male luer of a winged needle, which may be connected to the counterpart male luer by means of a tubing, or a catheter. The winged needle and the catheter may each be inserted to the patient, for example a vein of the patient. As with the needle assembly 30 shown in FIG. 1, a cannula (not shown) protected by an elastomeric or rubber shroud 52s, extends into the cavity of body 52a. Same as with the vacutainer holder shown in FIG. 1, an opening 52o at the proximal end of the cylindrical hollow body 52a allows a fluid store such as the vacuum tube holder 46 shown in FIG. 1, to be inserted into holder 52a to establish a through passage between the catheter or winged needle, which may be connected to luer 52l via a tubing, and the interior of the fluid store. As noted above, both holders 52 and 54 are specially designed with the female luer 52l and male luer 54l extending from their respective necks 52n and 54n.

Saf-T Holder® 54 has the same construction as holder 52, but for male luer 54l fixedly extending from neck 52n of cylindrical hollow body 54a. Male luer 54l is adapted to be mated to a counterpart female luer that in turn may be connected to a winged needle, by means of a tubing, or a catheter.

Saf-T Holders® 56 and 58 have shorter, but wider cylindrical hollow bodies 56a and 58a than those of holders 52 and 54. As shown, holder 56 has a male luer 56l fixedly extending from its neck 56n while holder 58 has a female luer 58l fixedly extending from its neck 58n. As discussed above with respect to holders 52 and 54, the male luer 56l of holder 56 is matable to a counterpart female luer that may be connected to a winged needle via a tubing, or a catheter for interaction with a patient. Likewise, female luer 58l of holder 58 is adapted to be connected to a counterpart male luer that may be connected to a winged needle by means of a tubing, or a catheter for interaction with a patient.

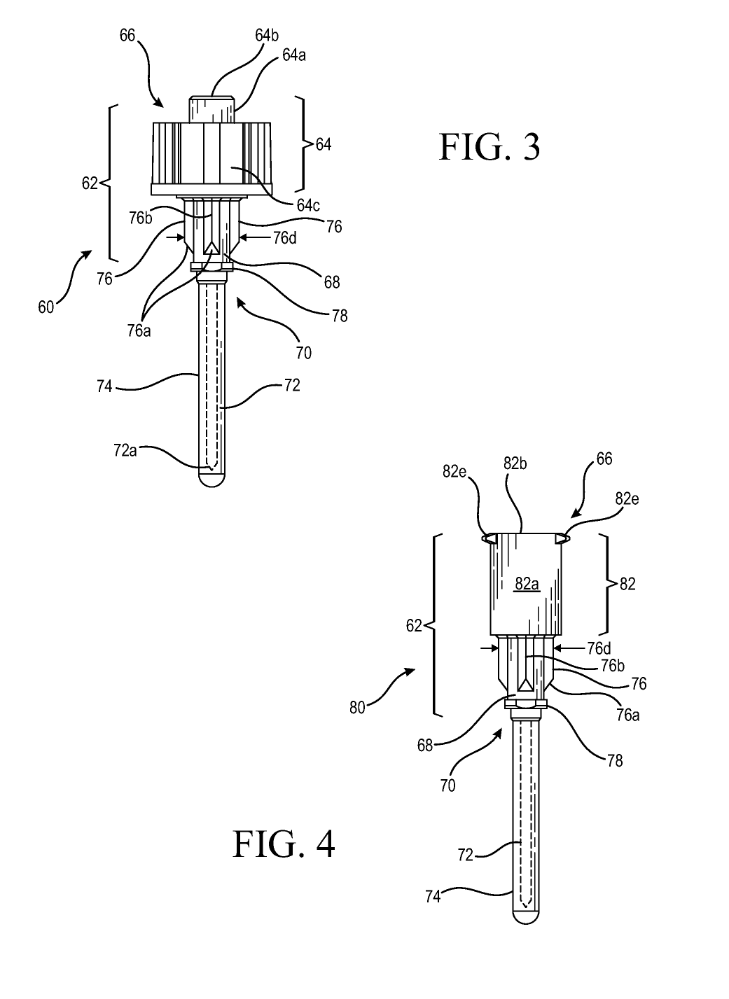

With reference to FIG. 3, a male luer adapter fitting 60 of the instant invention is shown to have a body 62 having a male luer 64 at a distal or one end 66, and an elongate cylindrical insert 68 extending away from male luer 64 toward a proximal end 70 of the fitting 60. Male luer 64 has a hollow elongate tapered center protrusion 64a that has an opening 64b that leads to a passage through luer 64. For the instant invention, the through passage extends along male luer 64 into the elongate cylindrical insert 68, to proximal end 70 and from there to a needle or cannula 72, shown in dotted line, so that a through passage extends from opening 64b at male luer 64 to the tip 72a of needle 72. As shown, needle 72 is enveloped by an elastomeric or rubber shroud 74, which is pushed backwards toward the elongate insert 68 by the septum of a fluid store tube, such as a vacuum tube, when in use as described with respect to the vacutainer holder shown in FIG. 1.

A plurality of ribs 76 each extend longitudinally along the elongate insert 68, at at least a given length that enables the ribs to fully engage with the internal threads formed at the inner circumferential wall of the neck of the tube holder, as will be explained in greater detail, infra. Each of ribs 76 has a tapered end 76a for ease of insertion. Also, the longitudinal sides of each rib are angled away from the insert body to converge or meet at an apex 76b, which forms a sharp edge for each of the ribs. At or proximal to proximal end 70 there is a flange or rib 78 integrally extending from elongate insert 68. Flange 78 may be a continuous circumferential flange or may be discontinuous extension portions that circumferentially formed about the proximal portion of the insert. Discontinuous or not, flange 78 acts as a locking mechanism to fixedly couple fitting 60 to the vacuum tube holder, as will be described in greater detail, infra.

FIG. 4 shows a female luer adapter fitting 80 of the instant invention. Elements of the female adapter fitting 80 that are the same as the male luer adapter fitting 60 of FIG. 3 are labeled the same. The major difference between the female luer adapter fitting of FIG. 4 and that of the male luer adapter fitting in FIG. 3 is the luer. As shown, the female luer adapter fitting 80 has a female luer 82 that is a part of body 62. Female luer 82 has a body portion 82a that has a number of tabs or ears 82e that enable female luer 82 to mate with a counterpart male luer, which has an outer shroud with an internal thread that mates with ears 82e. Such shroud is exemplified by shroud 64c of the male luer 64 shown in FIG. 3. Although not shown, an opening 82b is provided at the open end of female luer 82 to form a through passage along body 62, and needle 72. As with the male luer adapter fitting shown in FIG. 3, the female luer adapter fitting 80 of FIG. 4 also has an elongate insert 68 with a plurality of ribs 76 extending longitudinally along insert 68 for a given distance. The construction of inserts 68 for the female luer adapter fitting 80 of FIG. 4 and the male luer adapter fitting 60 of FIG. 3 is the same. With the plurality of ribs 76 spatially circumscribing insert 68, a circumferential dimension is formed by the respective edges 76b of ribs 76. This circumferential dimension may be represented by the diameter 76d, as per shown in both FIGS. 3 and 4.

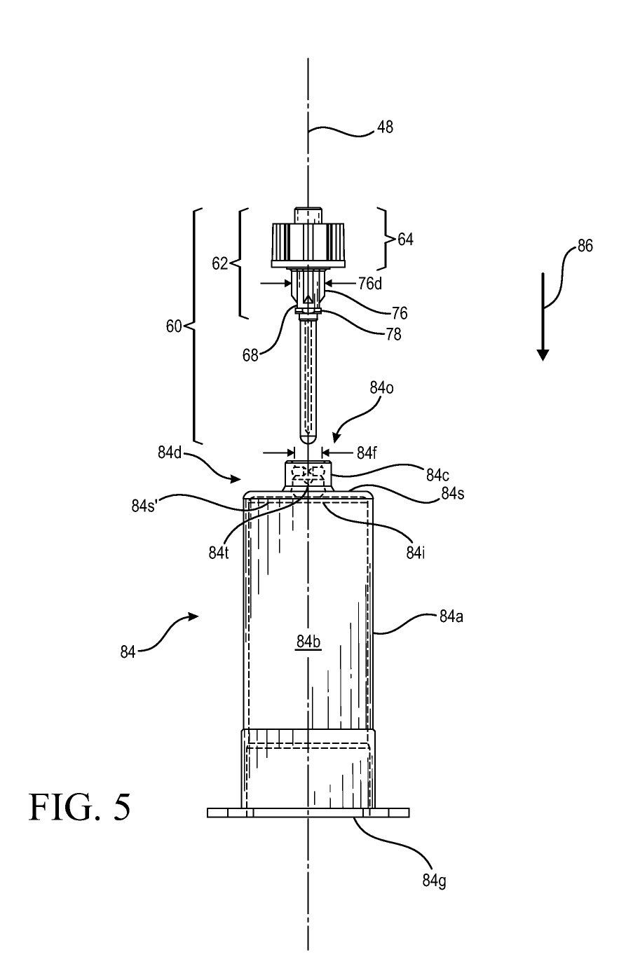

FIG. 5 shows a luer fitting adapter, in this instance a male luer fitting adapter as discussed above with respect to FIG. 3, in alignment along longitudinal axis 48 with a fluid store holder 84, which may be same or similar to the vacuum tube holder 2 shown in FIG. 1, sans housing 20 attached thereto. As shown, holder 84 has a hollow body 84a forming a cavity 84b. A neck 84c integrally extends from body 84a at the distal receptacle, or one end 84d of body 84a. Given that neck 84c has a smaller circumference than body 84a, a circumferential shoulder 84s is formed at the junction where neck 84c meets the distal end 84d of body 84a. As with the vacuum tube holder 2 of FIG. 1, neck 84c has a threaded through bore 84t with an opening 84o. Through bore 84t is formed by threads along the inner circumferential wall of neck 84c, and is deemed to have a circumferential dimension that defines an internal diameter 84f for neck 84c.

For the instant invention, the circumferential dimension 76d formed by the ribs 76 (or more precisely the longitudinal sharp edges 76b of the ribs) at cylindrical insert 68 of the luer adapter fitting is designed to be sufficiently larger than inner circumferential dimension 84f formed at through bore 84t, so that when fitting 60 is coupled to holder 74, by press-fitting insert 68 to through bore 84t, ribs 76, due to their respective sharp edges 76b, would cut or score lengthwise the internal threads at through bore 84t. Once fitting 60 is fully coupled to holder 84, with insert 68 fully press-fitted to neck 84c in the direction as indicated by directional arrow 86, the respective sharp longitudinal edges 76b of ribs 76 are frictionally scored longitudinally along the threads internally formed in through bore 84t. As a consequence, anti-rotation between the fitting and the holder is achieved as relative rotational movement between the luer 60 and holder 84 is prevented, so that a unified rotational movement is effected, when either fitting 60 and/or holder 84 is rotated in a given direction. Also, the forced fitting of insert 68 causes the ribs 76 to frictionally held by the scored portions of the internal threads at neck 84c so that fitting 60 is frictionally coupled to holder 84, absent a deliberate force to remove fitting 60 from holder 84 in a direction counter to that shown by directional arrow 86.

To prevent fitting 60 from being removed or separated from holder 84, the circumferential flange 78, or dis-continuous flange extensions, formed at the proximal end of insert 68 would latch onto the internal shoulder 84s′ at the base of through bore 84t (at an internal space 84i formed thereat), when insert 68 is fully press-fitted to neck 84c. That flange 78 is able to act as a catch is due to the elastic property of the medical plastics material used to make the fitting, which allows flange 78 to be compressed, as it passes through through bore 84t, and returns to its original position, once it passes into the space 84i at the internal shoulder 84s′ at holder 84. Once flange 78 latches onto internal shoulder 84s′ and is positioned at space 84i, fitting 60 is fixedly coupled to holder 84. A reception opening 84g at the proximal end of holder 84 allows a fluid tube holder such as the vacuum tube holder 46 (FIG. 1) to be inserted into cavity 84g to establish a fluid path with luer 60.

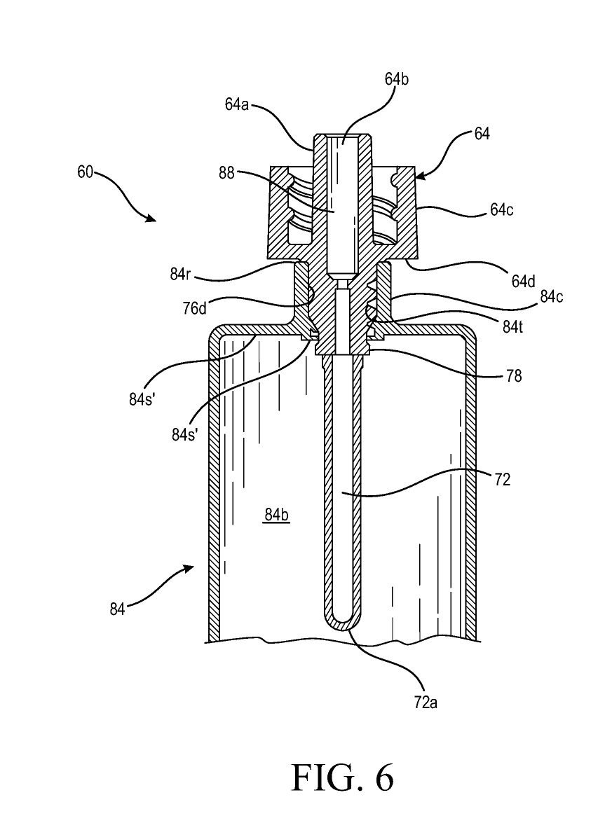

FIG. 6 shows an enlarged cross-sectional view of the inventive apparatus where fitting 60 is fully coupled to holder 84. As shown, the outer shroud 64c of the exemplar male luer adapter fitting 64 has its base 64d resting on the top rim 84r of neck 84c of holder 84. The cross-sectional view also shows an edge 76d of one of the plurality of ribs 76 in contact relationship with the longitudinal section of the internal circumferential wall of neck 84 due to edge 76d scoring the threads at the internal circumferential wall of neck 84c. Some of the internal threads 84t are shown at the right-hand side, with reference to the drawing, at the inner circumferential wall of neck 84c. As further shown, fitting 60 is non-removably coupled to holder 84, due to flange 78 having latched to the internal shoulder 84s′ of holder 84. Thus coupled, a through passage 88 extends between opening 64b at luer fitting 60 to the interior cavity 84b of holder 84 by way of needle 72, which conventionally has an aperture at tip 72a. Needle 72 is insertable to a fluid store such as the vacuum tube holder 46 shown in FIG. 1. Although not shown, a counterpart luer, in this instance a female luer, is connectable to male luer fitting 60, so that a fluid path may be established between the winged needle or catheter that is connected to the counterpart female luer and the interior cavity of the vacuum tube holder of the exemplar device of FIG. 6.

Instead of the male luer adapter fitting as per shown in FIG. 6, the holder 84 may have coupled to its threaded through bore 84t the elongate cylindrical insert of a female luer adapter fitting, such as fitting 80 shown in FIG. 4. In that instance, the female luer of the fitting is matable to a counterpart male luer.

Thus, with a fluid store holder that has a neck having an internally threaded through bore, different types of devices may be manufactured using the same holder. For example, a holder with a threaded through bore at its neck can be used to accept a double ended needle assembly such as that shown in FIG. 1, so that a vacutainer holder device as discussed with respect to FIG. 1 may be configured. On the other hand, the same holder may be used to configure either of the Saf-T Holder® devices shown in FIG. 2, depending on whether the male luer adapter fitting shown in FIG. 3 or the female luer adapter fitting shown in FIG. 4 is coupled to the holder. As a consequence, the cost for manufacturing different types of devices that may be used for transferring fluid between a patient and a fluid store is reduced, as the same fluid store holder may be used for all those devices. Thus, so long as the tube holder has a threaded internal bore and the luer fitting has an insert that has score mechanisms such as the afore-discussed longitudinal ribs that act as anti-rotation means and a lock mechanism such as a flange, rib or circumferentially formed extensions that would snap lock to the base opening of the through bore, cost effective fluid transfer devices may be manufactured simply.

Inasmuch as the present invention is subject to many variation, modifications and changes in detail, it is intended that all matter described throughout this specification and shown in the accompanying drawings be interpreted as illustrative only. For example, the configured blood drawing or fluid transfer device in FIG. 6 is shown without a needle protection housing, such as the housing 20 shown attached to holder 2 in FIG. 1. However, it should be appreciated that the device of FIG. 6 may have added thereto the needle protection housing shown in FIG. 1 by providing a circumferential boss externally about neck 84c of holder 84 to which a base of a needle protection housing as discussed in FIG. 1 may be rotatably connected. Further, it should be appreciated that the circumferential catch flange 78 may be formed at any portion along the distal end portion of insert 68, so long as that flange is latchable to the underside shoulder portion of the internal shoulder of the fluid store holder. A space may be formed at the interior base of the shoulder of the holder to accept the flange. Accordingly, it is intended that the invention be limited only be the spirit and scope of the hereto appended claims.

1. A fitting adapted to be attached to a fluid store holder having a cylindrical body including one end with a neck having a threaded through bore with internal threads and an other end having an opening adapted to receive a fluid store, comprising:
a body having a luer at one end and an elongate cylindrical insert extending away from the luer to an other end, a plurality of ribs spatially formed circumferentially about the insert with each rib extending longitudinally along at least a given length of the insert, the circumferential dimension formed by the plurality of ribs being larger than the circumferential dimension of the threaded through bore so that when the insert is press fitted to the through bore, the ribs would score the threads along the through bore to prevent relative rotation between the fitting and the holder.

2. Fitting of claim 1, wherein the insert includes a catch that engages with the internal shoulder of the wall of the fluid store holder that forms the base of the through bore along the neck of the fluid store holder so that once the fitting is fully inserted into the through bore, the catch latches onto the internal shoulder at the base of the neck to prevent the fitting from being removed from the fluid store holder.

3. Fitting of claim 2, wherein the catch comprises a circumferential flange about the insert, the flange having a diameter greater than the diameter of the internal shoulder so that the flange snap fits over the internal shoulder when the insert is fully inserted to the through bore to thereby fixedly hold the connector to the tube holder.

4. Fitting of claim 1, wherein the luer of the fitting comprises a male luer.

5. Fitting of claim 1, wherein the luer of the fitting comprises a female luer.

6. Fitting of claim 1, wherein the fitting further comprises a cannula extending from the end of the insert, a through passage extending along the body from the one end through the insert so that fluid is passable between the cannula and the luer when the fluid store is received into the cylindrical body of the holder.

7. Apparatus, comprising:
a fluid store holder having a cylindrical body including one end with a neck having a through bore threaded with internal threads and an other end having an opening adapted to receive a fluid store;
a fitting coupled to the neck of the tube holder, the fitting including a luer at one end and an elongate cylindrical insert extending away from the luer to an other end, a plurality of ribs spatially formed circumferentially about the insert with each rib extending longitudinally along at least a given length of the insert, the circumferential dimension formed by the plurality of ribs being larger than the circumferential dimension formed by the threaded through bore, the insert press fitted to the through bore with the ribs scoring the threads along the through bore to prevent relative rotation between the connector and the holder.

8. Apparatus of claim 7, wherein the fitting further comprises a flange extending about the insert, the flange having a diameter greater than the diameter of the internal shoulder formed at where the through bore and the cylindrical body meet so that once the insert is fully fitted to the through bore, the flange catches onto the shoulder to thereby fixedly hold the connector to the tube holder.

9. Apparatus of claim 7, wherein the holder is a vacuum tube holder and the fluid store is a vacuum tube.

10. Apparatus of claim 7, wherein the luer at the one end of the fitting comprises a male luer.

11. Apparatus of claim 7, wherein the luer at the one end of the fitting comprises a female luer.

12. Apparatus of claim 7, wherein the fitting further comprises a cannula extending from the end of the insert, a through passage extending from the one end of the fitting through the insert so that fluid is passable between the cannula and the luer at the one end when a fluid store is received into the cylindrical body of the holder.

13. Apparatus of claim 7, further comprising a needle protection housing having a collar rotatably mounted to the neck of the holder, the housing being pivotable relative to the collar so that the housing is pivotally connected to the holder.

14. Method comprising the steps of:
providing a fluid store holder having a cylindrical body including one end with a neck having a threaded through bore with internal threads and an other end having an opening adapted to receive a fluid store;
providing a fitting including a luer at one end and an elongate cylindrical insert extending away from the one end to an other end, a plurality of ribs spatially formed circumferentially about the insert with each rib extending longitudinally along at least a given length of the insert, the circumferential dimension formed by the plurality of ribs being larger than the opening of the threaded through bore at the neck of the holder; and
inserting the fitting to the through bore so that the ribs score into the threads along the through bore to prevent relative rotation between the fitting and the holder so that the luer at the one end of the fitting is adapted to receive a counterpart luer.

15. Method of claim 14, further comprising the step of:
forming a flange about the insert to have a diameter greater than the diameter of the internal shoulder formed at where the through bore and the cylindrical body meet; and
fully fitting the insert to the through bore to lock the flange onto the shoulder to thereby fixedly hold the connector to the tube holder.

16. Method of claim 14, further comprising the step of:
extending a cannula from the end of the insert to establish a through passage from the one end of the fitting through the insert so that fluid is passable between the cannula and the luer at the one end when a fluid store is received into the cylindrical body of the holder.

17. Method of claim 14, further comprising the step of:
forming the luer at the one end as a male luer.

18. Method of claim 14, further comprising the step of:
forming the luer at the one end as a female luer.

19. Method of claim 14, further comprising the step of:
rotatably mounting a collar of a needle protection housing to the neck of the holder, the housing being pivotable relative to the collar so that the housing is pivotally connected to the holder.
